(19) United States
(12) Patent Application Publication
(10) Pub. No.: US 2025/0210168 A1
(43) Pub. Date: Jun. 26, 2025

(54) DEVICES, SYSTEMS, AND METHODS FOR ELICITING BEHAVIORAL CHANGE

(71) Applicant: High Walk Ventures, Inc., Shakopee, MN (US)

(72) Inventors: Kimmo Antero Kyllonen, Shakopee, MN (US); Juha Einari Hietasarka, Tampere (FI); Eva Susanne Blazek, Pine Island, MN (US)

(73) Assignee: High Walk Ventures, Inc., Shakopee, MN (US)

(21) Appl. No.: 19/000,471

(22) Filed: Dec. 23, 2024

Related U.S. Application Data
(60) Provisional application No. 63/614,197, filed on Dec. 22, 2023.

(51) Int. Cl. G16H 20/00

(52) U.S. Cl. CPC G16H 20/00

(57) ABSTRACT

The present invention provides physical and digital games, puzzles, utensils, and systems capable of facilitating cognitive development and behavioral change. In one example, such systems provide digital real-time feedback while being able to simultaneously adapt to a player's skill level and cognitive abilities. Such feedback may include fixed or intermittent messages infused with a desired behavioral change, encouragement, or other forms of support. Through completion of the activity and positive reinforcement messages, a user may build self-confidence, spatial reasoning skills, and critical thinking skills.

### CROSS-REFERENCE TO RELATED APPLICATION

This application claims priority from U.S. Provisional Application Ser. No. 63/614,197, filed Dec. 22, 2023 and entitled “DEVICES, SYSTEMS, AND METHODS FOR ELICITING BEHAVIORAL CHANGE”, the disclosure of which is incorporated herein by reference.

### TECHNICAL FIELD

This disclosure relates to devices, systems, and methods for eliciting behavioral change. More specifically, the disclosure relates to physical and digital puzzles, games, utensils, and systems for eliciting behavioral change while providing users with a continuous learning experience.

### BACKGROUND

It is essential for a child, teenager, or adult to develop proper cognitive functioning or positive behavioral traits. Traditionally, depending on the individual, different tactics or activities may be implemented. For example, parents often provide children with one or more types of games or tasks to facilitate or otherwise aid in their cognitive development. Such games may range from simple puzzles and card games such as “Go Fish” to more complex puzzles and games such as chess, crossword puzzles, and word searches. However, such games often lack complexity and interactivity. While they may be useful for developing spatial awareness and some form of cognitive functioning, such games often lack the capacity to adapt to varying skill levels, provide feedback, or furnish users with an ongoing learning experience. Further, existing games and puzzles do not effectively bridge the gap between physical and digital gameplay, limiting their appeal and educational potential in a technology driven age.

For example, in the traditional Chinese dissection puzzle, “Tangram”, seven puzzle pieces are provided in the form of two large triangles, one medium-sized triangle, two small triangles, one square, and one parallelogram. These pieces, known as “tans”, are flat, two-dimensional shapes. The primary objective of Tangram is to rearrange the tans to form a specific shape, using all the pieces, without incurring any overlap. The shapes formed by the tans are general silhouettes or outlines and can range from simple geometric figures to more complex shapes resembling animals, people, numbers, and objects. As with other common games, while Tangram is a useful tool for developing individual problem-solving skills and spatial reasoning, it possesses certain limitations in the way of creativity, collaboration, persistence, and determination due to its tangible and single-player nature, lack of connected resources, and inability to adapt in real-time to a user's skill set. Moreover, although the shapes are in the square root of two relative to each other, the limited number of shapes only allow for a half pattern and don't give a user full understanding.

Additionally, as adults, various activities in the form of cooking and writing are often undertaken to develop positive behavioral characteristics. For example, adults may engage in various writings tasks and cooking challenges to facilitate or otherwise develop positive behavioral characteristics. However, many of these tasks suffer from the same deficiencies as noted above. Specifically, such activities often lack the ability to adapt to varying skill levels, provide feedback, or furnish users with an ongoing learning experience. Further, these challenges often do not effectively bridge the gap between physical and digital examples.

Therefore, what is needed are digital and physical games, puzzles, objects, utensils, and applications capable of remedying these deficiencies while aiding in a user's cognitive development.

### SUMMARY

The present invention provides physical and digital games, puzzles, utensils, and systems capable of facilitating cognitive development and behavioral change. In one example, such systems provide digital real-time feedback and encouragement while being able to simultaneously adapt to a player's skill level and cognitive abilities. Such feedback may include fixed or intermittent messages infused with a desired behavioral change, encouragement, or other forms of support. Through completion of the activity and positive reinforcement messages, a user may build self-confidence, critical thinking skills, and other behavioral traits.

In one example, the present invention includes an interactive system with physical and digital components. Such physical and digital components may include physical puzzle pieces with a wireless communication interface, e.g., NFC tags, Bluetooth, RFID, etc., for interacting with a digital behavioral modification system. Such system may generate and display a desired puzzle configuration, a real-time image of the physical pieces, or supportive feedback and encouragement. Once the pieces are arranged in a desired order, a user may share, download, or otherwise communicate their creation with others through the system.

In other examples, the physical and digital components may include utensils, e.g., cooking utensils, writing utensils, coat hook etc., with a wireless communication interface for communicating with a behavioral modification system. Depending on the type of physical component, the system may display recipes, stories, feedback, chores, tasks, or any other information to encourage or otherwise assist the user in completion of the activity and, in turn, elicit positive behavioral change.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 illustratively shows an interactive system in accordance with examples of the present invention.

FIG. 2 illustratively shows a behavioral modification system in accordance with examples of the present invention.

FIG. 3 illustratively shows a puzzle game in accordance with an example of the present invention.

FIG. 4 illustratively shows puzzle game pieces in accordance with an example of the present invention.

FIGS. 5A-5D illustratively show puzzle piece patterns in accordance with examples of the present invention.

FIGS. 6A-6D illustratively show additional puzzle piece patterns in accordance with examples of the present invention.

FIG. 7 illustratively shows a board game layout in accordance with an example of the present invention.

FIGS. 8-11 illustratively show user interface displays of a behavior modification system in accordance with examples of the present invention.

FIG. 12 illustratively shows a utensil in accordance with an example of the present invention.

FIGS. 13-14 illustratively show a hanger or hook assembly in accordance with examples of the present invention.

FIG. 15 illustratively shows a method of displaying encouragement, nudge messages, feedback, or other information in accordance with an example of the present invention.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

In accordance with the present invention, an interactive system and method are provided for eliciting positive behavioral change and cognitive development. In one example, the interactive system includes physical components, e.g., physical puzzle pieces, game pieces, utensils, etc., with a wireless communication interface for communicating with a behavioral modification system. Behavioral modification system may take the form of a web-based application, artificial intelligence, neural network, or any other form of software or hardware capable of carrying out and performing the functions disclosed herein.

In operation, as a user completes a task with one or more physical objects, the behavioral modification system may generate and display nudge messages infused with behavioral change techniques related to the task. Through completion of the task, positive behavioral change and cognitive development may be elicited. In some examples, results may be shared with parents, grandparents, friends, or others through the system.

Stated differently, provided herein is an interactive system and method that uses creative and fun tasks as a carrier for nudge messages infused with behavior change techniques. Such systems and methods may include physical objects embedded with a wireless communication interface and a digital component in the form of a behavioral modification system. Such system may generate feedback or encouragement, create a digital mirror image or twin image, or furnish information based on the received information from the physical objects.

In one example, physical components of an interactive system include a puzzle embedded with a wireless communication interface for communicating with a behavioral modification system. In this example, the behavioral modification system may generate a digital twin or mirror image, provide encouragement, feedback, or other user-generated content during completion of the puzzle to elicit self-confidence or other positive behavioral traits. In turn, this may give users or their respective family members peace of mind. For example, using the puzzle as a creative and fun behavior change carrier, family members may feel confident that the puzzle will elicit the desired behavioral traits. Once completed, results may be communicated, downloaded, and shared with grandparents or other family members. Additionally, the behavioral modification system may also allow for a multi-player experience in which multiple users can compete or otherwise work together to build a displayed puzzle shape. However, other multiplayer modes may be selected as well, e.g., show down, grand competition, etc.

In another example, an interactive system includes physical objects in the form of utensils, e.g., cooking utensils. The utensils may be embedded with a wireless communication interface for communicating with a behavioral modification system. In this example, the behavioral modification system may display recipes, cooking tips, step-by-step instructions, and real-time feedback as a user cooks a meal. However, other user provided content may be generated and displayed as well. In turn, this may give a user self-confidence, encourage weight loss, and demonstrate proper calorie management using cooking as a creative and fun behavior change carrier. Additionally, the behavioral modification system may also allow for a multiplayer experience in which users race to cook a meal, e.g., show down, grant competition, etc., or otherwise provide helpful comments or feedback to each other.

In a further example, an interactive system includes physical objects in the form of writing instruments, e.g., pen, pencil, marker, etc. The writing instrument may be embedded with a wireless communication interface for communicating with a behavioral modification system. In this example, the behavioral modification system may generate and display stories, factual and misleading information, fables, mirror the physical writing of a user, provide feedback, etc. Based on the displayed information, a user may be prompted to sort through the facts and misinformation, modify, add to, or subtract from the information, or otherwise answer questions about the displayed material. Using story-telling as a creative and fun behavior change carrier, a user may develop self-confidence, critical thinking skills, and other positive behavioral traits. In this example, the behavioral modification system may also include a multi-player mode in which users are able to compete, e.g., via a show down mode, grand competition, etc.

In another example, an interactive system includes a physical object in the form of a hook assembly. A hook assembly may be embedded with a wireless communication interface for communicating with a behavioral modification system. In this example, when an object is placed on a hook assembly, the behavioral modification system may generate push notifications to notify a subscriber or other user of an object placed on the hook. In one example, this notification may indicate the presence of a visitor or resident at a subscriber's home or business. While a hook is illustratively provided, other items and objects may be used as well.

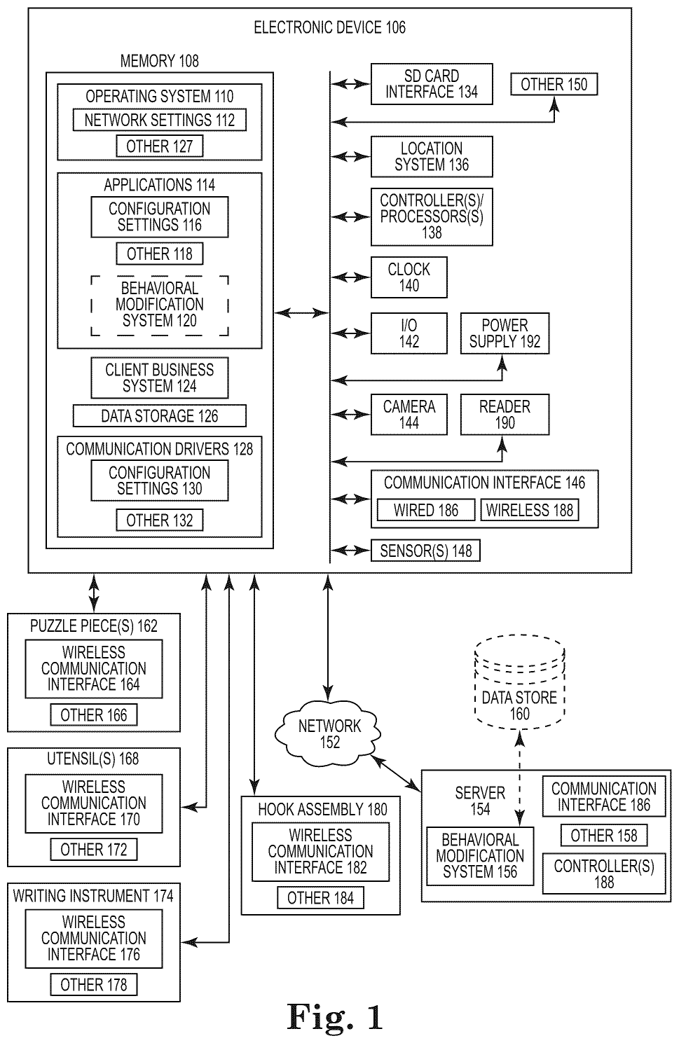

FIG. 1 illustratively shows an interactive system in accordance with one example of the present invention. The interactive system illustratively includes puzzle pieces 162, cooking utensils 168, pens 174, and a hook assembly 180 with respective wireless communication interfaces 164, 170, 176, and 182 for communicating with electronic device 106 and behavior modification system(s) 156/120. While four types of objects are illustratively shown, it is to be understood that system 156/120 may operate with any one type of objects, e.g., puzzle 162, utensils 168, pens 174, or hook assembly 180, or alternatively, some or all of them. Additionally, while puzzle pieces 162, cooking utensils 168, writing instruments 174, and a hook assembly 180 are illustratively shown, other types of objects may be used as well. It is to be understood that these are examples only. Further, while behavioral modification system 156 is shown on server 154 accessible via network 152, in other examples, all, or parts, of behavioral modification system 156 may be stored on electronic device 106, e.g., as a system application 120. However, in other examples, system application 120 may be used to simply access the services of system 156 through server 154. Moreover, while a singular server 154 is shown, it is to be understood that behavioral modification system 156 may be located across multiple servers as well.

Through server 154, remote computation, software, data access, and storage services may be offered that do not require end-user knowledge of the physical location or configuration of the system. Remote server 154 may deliver services of system 156 over a wide area network, such as the internet, using appropriate protocols. In one example, system 156 may be accessed through a web browser or other computing component. Computing resources of a remote server may be consolidated at a remote data center or dispersed. Further, such infrastructure may deliver services through shared data centers while also appearing as a singular access point. However, regardless of where system 156/120 is stored, it is contemplated that such services may be accessed using electronic device 106.

Electronic device 106 may take the form of a handheld mobile computing device, tablet, or other computing device, e.g., iphone, ipad, laptop computer, desktop computer, etc. Device 106 may communicate with any or all of pieces 162, utensils 168, pens 174, or hooks 180 and simultaneously deploy or allow access to system(s) 156/120. As illustratively shown in FIG. 1, device 106 may include memory 108, an SD card interface 134, a location or positioning system 136, controllers/processors 138, a clock 140, input/output devices 142, a power supply 192, a camera 144, reader 190, communication interface 146, sensors 148, and other components 150 connected through a bus. Input/output devices 142 may include buttons, touch sensors, optical sensors, microphones, touch screens, displays, speakers, and printer ports. However, other input/output devices may be used as well. Memory 108 may include all types of tangible volatile and non-volatile computer-readable memory devices. Memory 108 may further include computer storage media.

In one example, memory 108 may store computer readable instructions, e.g., logic or modules, that, when executed by controller(s)/processor(s) 138, 332 causes controller(s)/processor(s) 138, 332 to perform computer-implemented steps or functions associated with system 156/120. Further, it is expressly contemplated that any or all the identified components of electronic device 106 may be used in accordance with system 156/120 to carry out the disclosed functions described herein. For example, positioning system 136 may include a global positioning system, a LORAN system, a dead reckoning system, a cellular triangulation system, or any other form of positioning system that generates positional information for system 120/156. However, this is a mere example only, and other components may provide additional information as well.

In operation, as a user carries out an activity with one of the physical objects shown in FIG. 1, behavioral modification system 120/156 may relay or otherwise display feedback on electronic device 106, e.g., through display of input/output device 142. The type of feedback may largely depend on the desired activity. For example, in the context of a puzzle, feedback may include displaying a digital twin image, delivering encouragement and feedback, or providing other forms of user provided content. As a user completes the physical puzzle in real time, behavioral modification system 120/156 may also simultaneously or automatically track a user's progress, update such displayed information, or display new information. Specifically, all or any individual piece of the puzzle may include a wireless communication interface, e.g., 164, 170, 176, or 182, allowing system 120/156 to digitally track and display a user's progress. As a user continues to complete the puzzle, system 120/156 may automatically update a user's progress, display new information, etc.

In the context of a cooking operation with utensils 168, such feedback from system 120/156 may include recipes, calorie tracking information, etc. As a user completes a meal or cooking operation, system 120/156 may display new information, encouragement, or other forms of feedback. In a writing application with writing instrument 174, system 120/156 may display stories, information/misinformation, fables, or other writing samples.

As discussed above, each physical object may include a wireless communication interface. The wireless communication interface may include near field communication (NFC) tags or any other form of communication component or protocol capable of relaying information about the physical objects, e.g., objects 162, 168, 174, or 180, to electronic device 106 and accessing system 120/156. For example, such wireless communication interface may include NFC tags, Bluetooth, WIFI, RFID, cellular, or satellite. However, if NFC tags are used, reader 190 of electronic device 106 may be used to subsequently power and obtain information from the tags, e.g., interfaces 164, 170, 176, and 182. Once information is obtained, system 120/156 may present a user with any or all the functionality of the system through a display of an electronic device as will be discussed below.

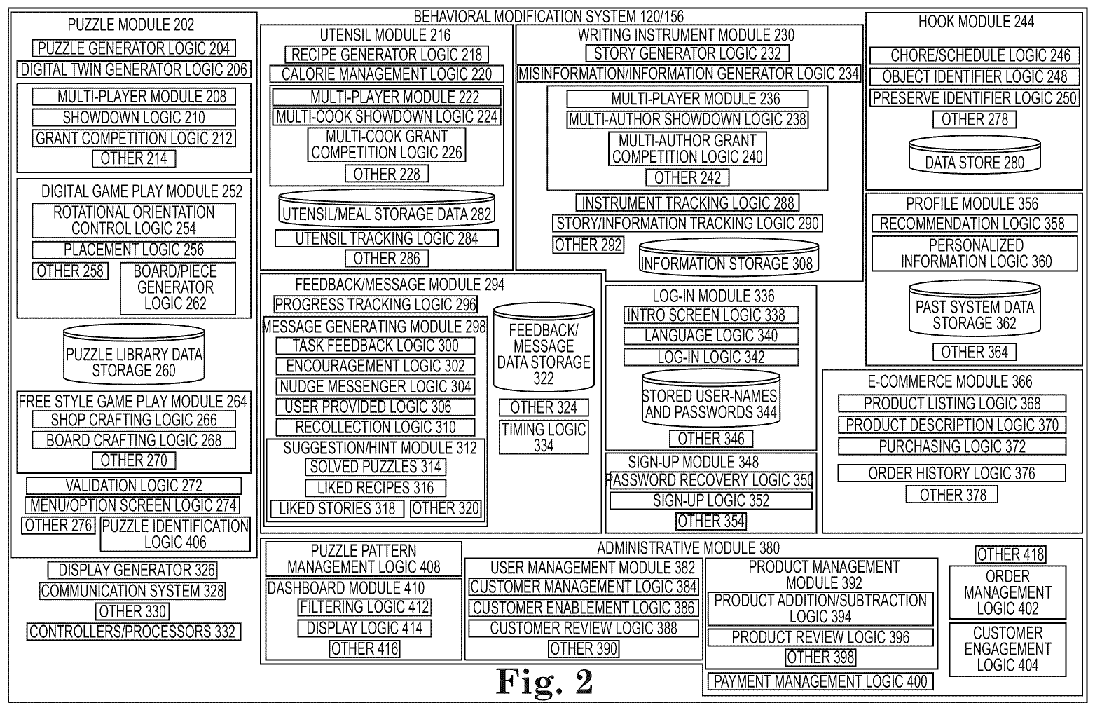

FIG. 2 illustratively shows a behavioral modification system in accordance with an example of the present invention, e.g., system 120/156. While system 120/156 illustratively includes a variety of different modules, logic, controllers/processors, non-transitory or transitory data storage devices, and display generator, it is to be understood that any or all the components of system 120/156 may be stored locally within an electronic device or remotely on a server. As shown, system 120/156 may include display generator 326 for generating a display on a display device, e.g., device 142, communication system 328 for allowing communication between components of system 120/156, electronic device, and server over a network, controller(s)/processor(s) 332, and a wide range of modules and logic for carrying out the functions and services described herein.

While many different types of modules and logic are shown, additional or alternative forms may be used as well. In one example, system 120/156 includes a neural network or artificial intelligence-type system. In this example, system 156 may be trained prior to deployment and include any number of layers, or logic, for carrying out the functions and services described herein. However, other forms of software and hardware may be used as well. Further, in some examples, logic and modules may take the form of computer readable instructions that, when executed by a controller/processor, cause the controller/processor to carry out the functions set forth herein. Specifically, depending on the type of physical object and communication interface, an electronic device, after actuating with a communication interface, may access and display different information and games based on the modules and logic of system 120/156, type of physical object, and unique bar code or identifier associated with each communication interface.

For example, system 120/156 may include a puzzle module 202 for creating and allowing for digital puzzle gameplay and feedback; a utensil module 216 for displaying and sharing recipes, calorie tracking information, and feedback; a writing instrument module 230 for creating, displaying, and sharing stories, tracking information/misinformation, articles, blogs, etc.; a hook module 244 for identifying a presence of a user based on a placement of clothing article(s) and, in turn, displaying schedules, feedback, and chore lists; feedback/message module 294 for providing feedback, encouragement, nudge messages, and other information during operation of one or more physical devices; an administrative module 380 for managing and controlling system 120/156; a user profile module 356 for maintaining, for example, a user database of recipes, puzzles, tasks, and chore lists; a log-in or authentication module 336 for managing access to system 120/156; and an e-commerce module 366 for purchasing completed puzzles and other completed works from system 120/156.

Beginning with puzzle module 202, module 202 may include various logic for generating, displaying, and allowing for personalized feedback and electronic gameplay on a display device, e.g., device 142. For example, module 202 may include puzzle pattern generation logic 204 for generating a digital puzzle pattern; digital twin or mirror generation logic 206 for identifying a unique serial number or identifier associated with a particular wireless communication interface, e.g., NFC tag, RFID, etc., and generating an identical digital replica of the puzzle on an electronic device display; validation logic 272 for identifying an acceptable placement of one or more puzzle pieces based on stored patterns; digital gameplay module 252 for controlling a placement of one or more digital puzzle pieces based on a selected pattern; free style module 264 for digitally creating puzzle patterns and shapes; multiplayer module 208 for permitting gameplay across multiple electronic devices; and non-transitory data storage 260 for storing completed or uncompleted puzzle patterns, shapes, etc.

Utensil module 216 may include recipe generator logic 218 for generating and displaying cooking recipes; calorie management logic 220 for tracking and displaying nutritional information of previously consumed food and recipes; multi-player module 222 for sharing recipes and engaging with other users during the cooking or preparation of a meal; utensil tracking logic 284 for identifying a particular serial number or identifier associated with the wireless communication interface of the utensil; non-transitory utensil/meal storage data 282 for storing nutritional information, recipes, previously cooked meals, etc.; and other logic 286.

Writing instrument module 230 may include story generator logic 232 for generating and displaying one or more user provided and uploaded stories, articles, etc.; misinformation/information generating logic 234 for generating and displaying information and misinformation parsable by a user; multiplayer module 236 for multiplayer story drafting competitions and exchanging user created stories, articles, etc.; instrument tracking logic 288 for identifying and recognizing a specific identifier associated with a physical writing instrument, e.g., pen, pencil, or marker; non-transitory instrument data storage 308 for storing previously completed and uncompleted stories, articles, blogs, etc.; and other logic 292.

Hook module 244 illustratively includes object identification logic 248 for identifying a presence of one or more clothing articles on a hook based on received information from a communication interface attached to a hook assembly; schedule/chore identification logic 246 for generating and displaying a task or chore list based on a detected presence of a clothing article; a non-transitory data store 280 for storing created tasks, chore lists, etc.; and other logic 278.

Feedback/message module 294 may periodically or continuously generate and display feedback, nudge messages, encouragement, or other information on a display device as a user interacts with physical objects, e.g., puzzle piece(s) 162, utensil(s) 168, instrument(s) 174, hook assembly 180, etc. As shown, module 294 includes progress tracking logic 296 for identifying a completion progress of one or more puzzle patterns, cooking recipes, tasks, or chore lists; timing logic 334 for identifying a passage of time as a user completes a puzzle, recipe, task, or chore list; a message generating module 298 for generating and displaying feedback, encouragement, nudge messages, memes, and other information based on received progress and timing signals from logic 296 and 334; non-transitory data store 322 for storing all feedback, encouragement, nudge messages, mirror image information, memes, and other information; suggestion/hint module 312 for generating and displaying hints, suggestions, or other useful information aimed at assisting a user with the completion of a puzzle, task, chore list, or recipe; and other logic 324.

User profile module 356 illustratively includes personalized information logic 360 that indexes, stores, produces, and displays all liked and completed puzzles, tasks, stories, chores, recipes, etc. for a particular user; recommendation logic 358 for analyzing indexed and stored puzzles, tasks, chores, and recipes and displaying recommended puzzles, tasks, chores, etc. based on any or all correlations between the completed activities, e.g., grilling recipes, oven recipes, yardwork, home renovation projects, animal-shaped puzzles, etc.; non-transitory user data storage 362 for storing indexed and completed tasks and puzzles, and other logic 364.

Log-in module 336 may include splash screen or introductory screen generation logic 338 for displaying a log-in, sign-up, or splash screen after actuation of a communication interface; language logic 340 for controlling a displayed language of words, phrases, and sentences; log-in logic 342 for checking and validating entered user credentials with those stored credentials for authentication purposes; non-transitory data storage 344 for storing usernames, passwords, and other user credentials; and other logic 346.

Sign-up or registration module 348 illustratively includes sign-up logic 352 for collecting registration information such as a user's name, password, email address, residential or commercial address, etc. prior to accessing system 120/156; password recovery logic 350 for generating a temporary password or username to assist a user in logging into system 120/156; and other logic 354.

E-commerce module 366 illustratively includes product listing logic 368 for listing available puzzles for purchase; product description logic 370 for displaying puzzle and author information; purchasing logic 372 for collecting and processing user payment information; order history logic 376 for tracking previously processed purchasing orders; and other logic 378.

Administrative module 380 may allow for administrative oversight and control over the various components of system 120/156. Module 380 may include puzzle pattern management logic 408 for uploading, modifying, and removing puzzles from a puzzle library; dashboard module 410 for generating and displaying an administrative dashboard; user management module 382 for managing, adding, and removing user profiles, reviews, and access to system 120/156; product management module 392 for adding, removing, and modifying listed puzzles for purchase; customer engagement logic 404 for sending message(s) to one or more customers or users; order management logic 402 for tracking user purchasing orders; and other logic. Such information may be stored in any or all data stores or mediums of system 120/156.

Stated differently, module 380 or system 120/156 may allow for certain administrative oversight. For example, an administrator may be able to manage all existing customers, enable/disable any customer from the application, review entire customer lists in detail, etc. Further, an administrator may be able to add products and review existing products and customer orders in detail. All forms of payment may also be reviewed. As it relates to the puzzle library, an administrator may be able to manage the library and accept or reject requests for new puzzles submitted by users or third parties. Additionally, administrators may be able to manage and upload additional or preexisting encouragement messages and acknowledgements. To cycle through each of these functions, a dashboard may appear. Such dashboard may allow for filtering of customers, game registrations, puzzle patterns, ordered puzzles, pending and completed orders, and pending and completed payments.

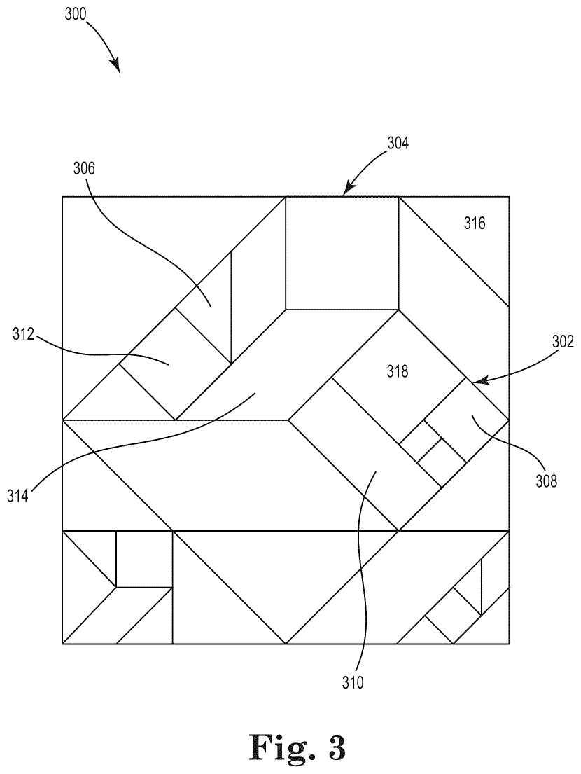

FIG. 3 illustratively shows a puzzle game in accordance with an example of the present invention. As shown, a puzzle game 300 (hereinafter referred to as “Irrational Squares”) may include a plurality of puzzle pieces 304, e.g., squares 312, triangles 306, parallelograms 314, etc., illustrating how two or more irrational ratios or numbers, e.g., √2, e^{x}, ϕ, etc. can complete each other's patterns. For example, as shown, pieces 304 may be sized and shaped based on the square root of two with one or more shapes 302 being further divided into squares 308 and rectangles 310 based on the golden ratio, phi (ϕ). Additionally, in some examples, any or all shapes 304 may include wireless communication interfaces 316, e.g., RFID tags, NFC tags, etc., for connecting with behavioral modification system 120/156.

In one example, the main objective of Irrational Squares 300 is to solve a puzzle by creating a pattern from a puzzle library. The puzzle may be created in many ways, and all are acceptable. Irrational Squares 300 may be played using physical pieces 304 or system 120/156. If physical pieces 304 are used, system 120/156 may manually or automatically generate and display a digital twin or mirror image of physical pieces 304 and the desired pattern or shape. However, other information relating to the pieces and desired pattern may be displayed as well.

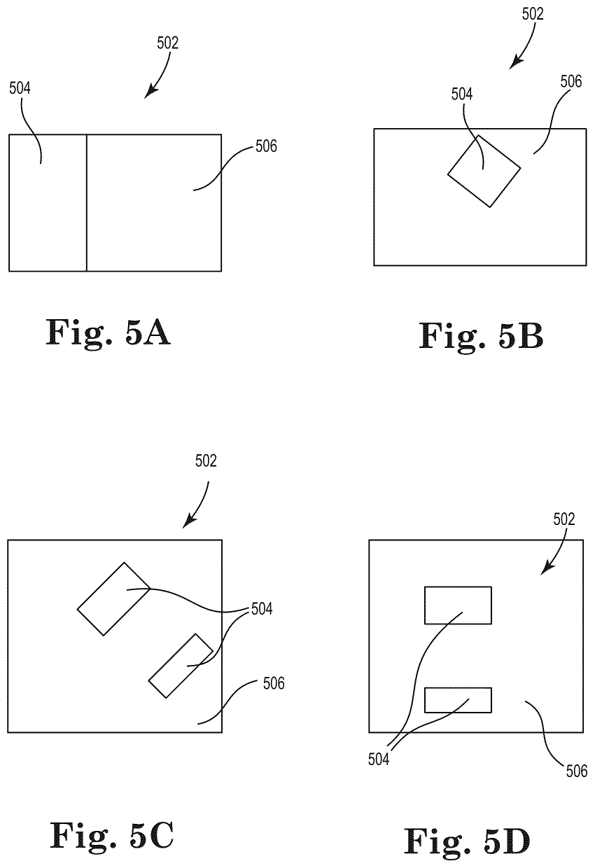

In Irrational Squares 300, a user may choose different puzzle pieces 304 and change their rotation and orientation to fit a desired pattern (as shown in FIGS. 5A-6D and FIG. 11). As users have varying skill levels, patterns may range from relatively simple to more complex. As a user successfully places pieces 304 to fit the pattern, system 120/156 may indicate correct placement of the piece and other helpful information as noted above. Once finished, system 120/156 may provide feedback in the form of a congratulatory prompt or notification.

In one example, there is no one way to solve any given pattern, making game 300 more interesting. Specifically, a user may solve the same pattern by placing different puzzle pieces 304 in various locations, creating a multitude of pattern possibilities, within the same pattern. For example, different square pieces can be in different locations on the puzzle board, e.g., in the center, edge, corner, etc.

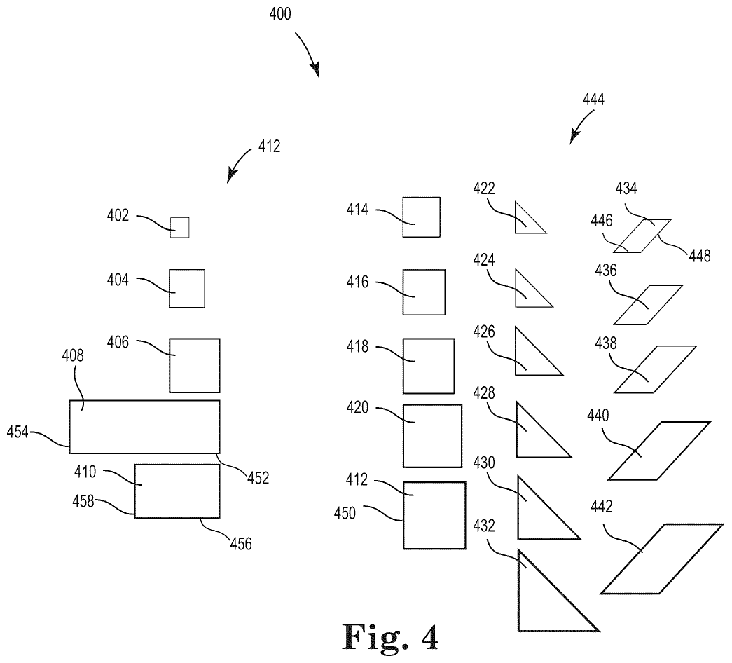

FIG. 4 illustratively shows puzzle game pieces in accordance with an example of the present invention. In one example, Irrational Squares 400 includes a square cut into ten or more distinct geometric pieces 444, e.g., 10 pieces, 20 pieces, 30 pieces, etc., based on one or more irrational ratios or ratios. For example, as shown, pieces 444 may include one large triangle 432, one medium-large triangle 430, one medium triangle 428, one medium-small triangle 426, one small triangle 424, one mini triangle 422, one large parallelogram 442, one medium-large parallelogram 440, one medium parallelogram 438, one medium-small parallelogram 436, one small parallelogram 434, one large square 412, one medium-large square 420, one medium square 418, one medium-small square 416, and one small square 414. Large square 412 may be further divided into one medium square 406, one small square 404, one mini square 402, one medium rectangle 408, and one mini rectangle 410.

However, it is to be understood that Irrational Squares 400 may include any number of pieces in accordance with the mathematical relationships described herein. For example, as shown in FIG. 3, a total board game area may take the form of a singular square with side lengths each equal to 8a√2. Of note, in one example, variable “a” equals a side length of the smallest square. All puzzle pieces may be dimensioned using variable “a” as a unit of measure. In one example, “a” may equal 22.45 mm so that each side length of a puzzle equals approximately 10 inches with a total area of approximately 64,512 mm² or approximately 2540 inches². However, “a” may equal other numerical values as well.

With side lengths of 8a√2, a square puzzle board area may be equal to (8a√2)². In this example, there may be 5+15 different puzzle pieces. A total puzzle piece count may equal 30. In this example, there is no one right way to solve the puzzle. Further, an area of different color puzzle pieces may be divided by a total area of the puzzle, e.g., ⅛=(a*4)\hat{ }2/(a*√2*8)\hat{ }2.

Each of the pieces 444 may be mathematically related such that, starting with the smallest shape, two of the smallest shapes create the next largest shape (e.g., two mini triangles create one small triangle). This may hold true for every shape except squares. For squares, the diagonal of the smallest shape may be the length of the next shape size. To indicate a mathematical relationship between the pieces, the pieces or board may be two or more colors.

For example, Irrational Squares 400 can demonstrate a full pattern with eight edges all in relation to the square root of two in a complete 2D pattern, the golden ratio

$$( \frac{1 + √ 5}{2} ) ,$$

and other shapes as irrational. The square root of two puzzle pieces 412-442 may be one color and the golden ratio pieces 402-410 another color. Additionally, game 400 may also show how irrational ratios can work together within the same puzzle. For example, golden ratio puzzle pieces 402-410 may be part of the square root of two puzzle and the square root of two puzzle pieces 412-442 may complete the golden ratio puzzle. This may lead to unexpected and surprising new patterns.

For example, in the square root of two puzzle, beginning with the smallest square 414, square 414 may include side lengths each equal to “a”. In turn, side lengths of a subsequently larger square may each equal the diagonal of the preceding square. Diagonals may be determined in accordance with the following equation:

$$Diagonal ( D ) = a √ 2$$

In this example, side lengths of medium-small square 416 may each equal a√2, i.e., (a*√2), side lengths of medium square 418 may each equal 2a, i.e., (a√2*√2), side lengths of medium-large square 420 may each equal 2a√2, i.e., (2a*√2), and side lengths of large square 412 may each equal 4a, i.e., (2a√2*√2). A total area of each square 412-420 may be determined based on the following equation:

$$Area ( A ) = base \times height$$

As such, a total area of smallest square 414 may equal (a)², an area of medium small square 416 may equal (a√2)², an area of medium square 418 may equal (2a)², an area of medium-large square 420 may equal (2a√2)², and largest square 412 may equal (4a)².

Right triangles 422-432 may follow a similar mathematical relationship. Specifically, beginning with mini triangle 422 with a base and height length each equal to “a”, a subsequent right triangle e.g., 424, may have a base and height each equal to the hypotenuse length of the preceding, smaller triangle. A right triangle hypotenuse may be determined in accordance with the Pythagorean theorem:

$${a}^{2} + {b}^{2} = {c}^{2}$$

For example, with a base and height each equal to “a”, a hypotenuse (c²) of mini triangle 422 may equal a√2, i.e., (a²+a²=c²). In turn, a base and height of the next largest right triangle 424 may each equal a√2 with a hypotenuse of 2a. Similarly, right triangle 426 may have a base and height each equal to 2a with a hypotenuse of 2a√2. Right triangle 428 may have a base and height each equal to 2 a√2 with a hypotenuse of 4a. Right triangle 430 may have a base and height each equal to 4a with a hypotenuse of 4a√2. Finally, right triangle 432 may have a base and height each equal to 4 a√2 with a hypotenuse of 8a.

Additionally, an area of triangles 422-432 may be determined in accordance with the following equation:

$$Area = \frac{1}{2} \times base \times height$$

As such, a total area of mini triangle 422 may be (a²/2); an area of small triangle 424 ((a√2)²/2), an area of medium-small triangle 426 ((2a)²/2), an area of medium triangle 428 ((2a√2)²/2), an area of medium-large triangle 430 ((4a)²/2)), and an area of large triangle 432 ((4a√2)²/2)).

For parallelograms 434-442, a similar relationship may be followed as each parallelogram 434-442 may be formed from two right triangles with one right triangle being inverted and connected to the other right triangle. Following the equations above, in this example, smallest parallelogram 434 may include a base 446 and height length each equal to “a” with a side length 448 of a√2. In turn, medium-small parallelogram 436 may include a base and height each equal to a√2 and a side length of 2a. Medium parallelogram 438 may include a base and height each equal to 2a and a side length of 2a√2. Medium-large parallelogram 440 may include a base and height each equal to 2a√2 and a side length of 4a. Finally, large parallelogram 442 may include a base and height each equal to 4a and a side length of 4a√2.

An area of parallelograms may be determined using the following equation:

$$Area ( A ) = base \times height$$

Following this equation, small parallelogram 434 may have an area of (a)². Medium-small parallelogram 436 may have an area of (a√2)². Medium parallelogram 438 may have an area of (2a)². Medium-large parallelogram 440 may have an area of (2a√2)² and large parallelogram an area of (4a)².

Following these mathematical relationships, a singular square with side lengths each equal to 8a√2 (as shown in FIG. 3) may be broken into one of pieces 402-410, one of pieces 414-420, two right triangles 422, three right triangles 424, three right triangles 426, four right triangles 428, one of right triangles 430 and 432, one of parallelograms 434-438 and 442, and three parallelograms 440.

Turning to golden ratio puzzle pieces 402-410, as shown, pieces 402-410 may be formed from the largest square 412 of the square root of two puzzle having side lengths 450 each equal to “4a”. In accordance with the present disclosure, seamless puzzles may be made with pieces having two or more dimensions with different ratios. For example, a two irrational ratio puzzle with side lengths of a and b may have:

$$a * √ 2 = b *$$

$$a = b * / √ 2$$

$$b = a * √ 2 /$$

Alternatively, a three irrational ratio puzzle with side lengths of a, b, and c may include:

$$a * √ 2 = b * = c * e$$

$$a = b * / √ 2 = c * e / √ 2$$

$$b = a * √ 2 / = c * e /$$

$$c = a * √ 2 / e = b * / e$$

$$( \text{?} number e = ( 1 \text{?} 1 / \text{?} = 2.7182618284590452353602874713526624977572470936 \text{?} 995 \text{?}$$

$$\text{?} \text{indicates text missing or illegible when filed}$$

Specifically, as shown in FIGS. 3 and 4, golden ratio pieces 402-410 may be formed from a square, e.g., square 302 and 412, of the square root of two puzzle. As noted above, in one example, square 412 of the square root of two puzzle may have side lengths 450 each equal to “4a”. To determine a side length of the largest golden ratio square piece 402, a total side length of square 412, e.g., “4a”, may be divided by the golden ratio,

$$( \frac{1 + √ 5}{2} )$$

so that a base and height length of largest golden ratio square 402 is each equal to

$$( \frac{8 a}{1 + √ 5} )$$

with a total square area of

$${( \frac{8 a}{1 + √ 5} )}^{2} .$$

To determine a side length of the next, or second, largest golden ratio square 404, a total side length of square 412, e.g., “4a”, may be subtracted by a side length of largest golden ratio square 402,

$$( \frac{8 a}{1 + √ 5} ) ,$$

so that a base and height of second largest square 404 is each equal to

$$( ( 4 a - ( \frac{8 a}{1 + \sqrt{5}} ) )$$

with a total square area of

$$( {( 4 a - ( \frac{8 a}{1 + \sqrt{5}} ) )}^{2} .$$

Turning to largest golden ratio rectangle 408, a length 452 of rectangle 408 may equal a total side length of square 412, “4a”. To determine a width 454 of rectangle 408, a total side length of square 412, “4a”, may be subtracted by the length of largest golden ratio square 402,

$$( \frac{8 a}{1 + √ 5} ) ,$$

so that width 454 of rectangle 408 equals

$$( ( 4 a - ( \frac{8 a}{1 + \sqrt{5}} ) )$$

and a total rectangle area equals

$$( 4 a x ( 4 a - ( \frac{8 a}{1 + \sqrt{5}} ) ) ) .$$

For smallest golden ratio square 406, a base and height of square 406 may each equal the total length of square 412, “4a”, subtracted by a length of the second largest square 404,

$$( ( 4 a - ( \frac{8 a}{1 + \sqrt{5}} ) ) ,$$

and width 454 of rectangle 408,

$$( ( 4 a - ( \frac{8 a}{1 + \sqrt{5}} ) ) ,$$

or stated differently,

$$( 4 a - 2 ( 4 a - ( \frac{8 a}{1 + \sqrt{5}} ) ) ) ,$$

so that a total area of square 406 equals

$${( 4 a - 2 ( 4 a - ( \frac{8 a}{1 + \sqrt{5}} ) ) )}^{2} .$$

Finally, for the smallest rectangle 410, a length 456 of rectangle 410 may equal the length of the smallest golden ratio square 406,

$$( 4 a - 2 ( 4 a - ( \frac{8 a}{1 + \sqrt{5}} ) ) ) .$$

In turn, a width 458 of rectangle 410 may equal a total length of square 412, “4a”, subtracted by the length of the smallest square 406,

$$( 4 a - 2 ( 4 a - ( \frac{8 a}{1 + \sqrt{5}} ) ) ) ,$$

and the length of the largest square 402,

$$( \frac{8 a}{1 + √ 5} ) ,$$

so that width 458 of smallest rectangle 410 equals

$$( 4 a - ( \frac{8 a}{1 + \sqrt{5}} ) - ( 4 a - 2 ( 4 a - ( \frac{8 a}{1 + \sqrt{5}} ) ) ) )$$

and a total area of smallest rectangle 410 equals

$$( 4 a - ( \frac{8 a}{1 + \sqrt{5}} ) - ( 4 a - 2 ( 4 a - ( \frac{8 a}{1 + \sqrt{5}} ) ) ) ) x ( 4 a - 2 ( 4 a - ( \frac{8 a}{1 + \sqrt{5}} ) ) ) .$$

However, different ratios don't have to be irrational. Additionally, each ratio may have different color puzzle pieces and include two or more puzzle pieces. In one example, each puzzle piece of the same color may be dimensioned using the ratio assigned to its color. Ratios may be applied to an individual puzzle piece's dimensions and relate to other pieces of the same color.

FIGS. 5A-5D illustratively show puzzle piece patterns in accordance with examples of the present invention. Specifically, as shown in FIGS. 5A and 5C-5D, a puzzle 502 may include square root of two puzzle pieces 506 capable of completing golden ratio puzzle patterns along with golden ratio pieces 504. Similarly, as shown in FIGS. 5B and 5C-5D, golden ratio puzzle pieces 504 can complete square root of two puzzle patterns along with puzzle pieces 506. In accordance with the present disclosure, it is expressly contemplated that pieces 504 and 506 may complete any number of patterns using any number of differently shaped pieces 504 and 506.

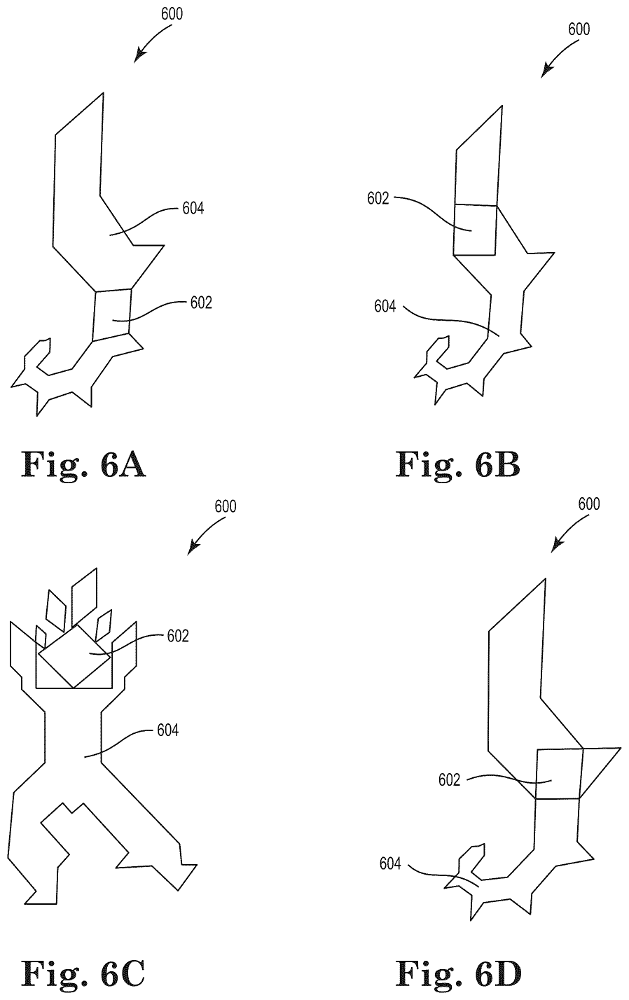

FIGS. 6A-6D illustratively show additional puzzle piece patterns in accordance with examples of the present invention. As shown, a puzzle game 600 may include square root of two pieces 604 and golden ratio pieces 602 completing different puzzle patterns. Pieces 602 and 604 may be placed in any number of different arrangements and orientations to complete a desired pattern. For example, as shown in FIG. 6A, golden ratio pieces 602 may complete a bottom half of a pattern, a middle portion of pattern as shown in FIG. 6D, or a top portion of a pattern as shown in FIG. 6B. To help show how pieces 602 and 604 may complete each other's patterns, pieces 602 and 604 may take any number of different colors, e.g., red, blue, black, green, etc.

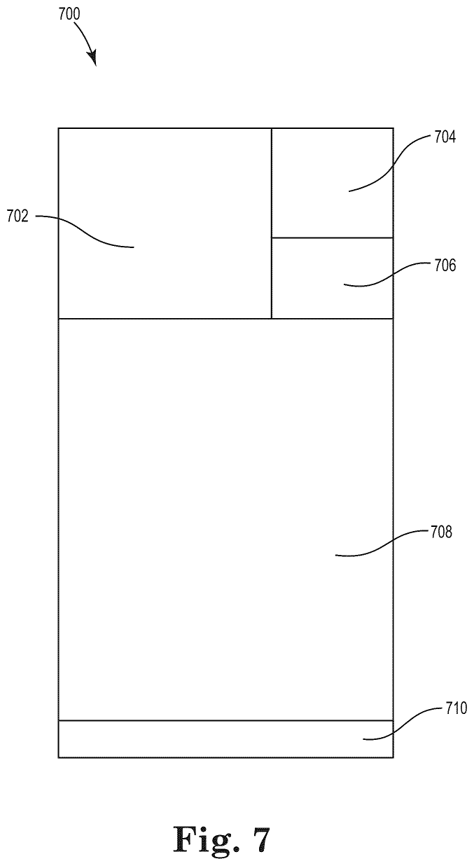

FIG. 7 illustratively shows a board game layout in accordance with an example of the present invention. As shown, a game board layout 700 may include a puzzle piece storage area 702 for storing and selecting puzzle pieces, an author profile section 704 for displaying author and pattern creation information, e.g., name, date, etc., a puzzle “story” section 706 setting forth different puzzle patterns, a game area 708 for arranging pieces in accordance with a desired pattern, and encouragement section 710 for displaying supportive feedback and information. A size of each section may vary depending on the sizes and shapes of the puzzle pieces. For example, for a total puzzle piece area of 100 inches, game area 710 may have a width of 20 inches and length of 26 inches. However, other sizes may be used as well.

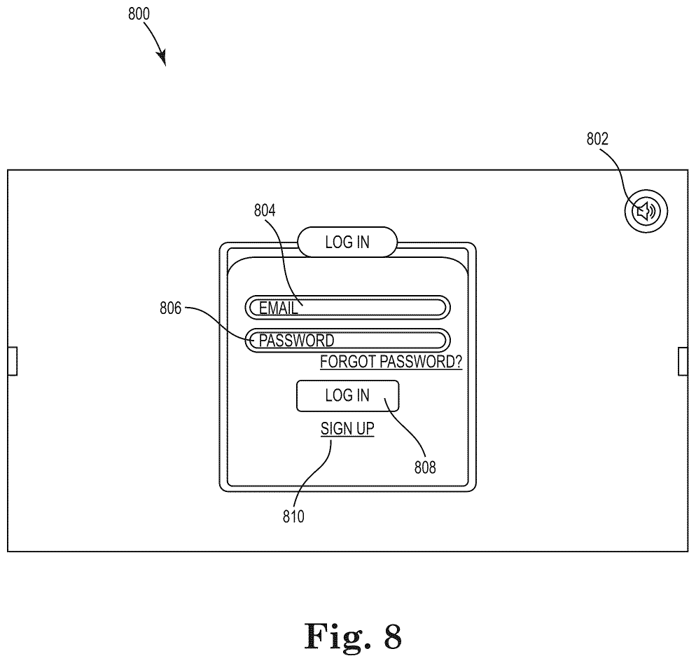

FIGS. 8-11 illustratively show user interface displays of a behavior modification system in accordance with examples of the present invention. As noted above, a physical object, e.g., puzzle piece, hook, utensil, writing instrument, etc., may include a wireless communication interface, e.g., NFC tag, RFID, Bluetooth, etc., that allows a user to connect to, and interact with, system 120/156. In one example, using an electronic device, e.g., device 106, a user may scan, activate, or otherwise read a communication interface on one or more physical objects to initiate interaction with system 120/156.

In one example, upon scanning or reading a communication interface, a user may be brought to a log-in screen as shown in FIG. 8. However, it's expressly contemplated that a user may be brought to other interfaces or screens as well. However, in this example, a log-in display 800 of system 120/156 may include an auditory icon 802 for generating any number of vocalized prompts, email address and password bars 804 and 806 for collecting a user's log-in credentials, and a sign-up icon 810 for collecting user information, e.g., name, address, date of birth, etc., prior to accessing system 120/156. While display 800 is in English, system 120/156 may present information in any other language as well. In one example, all users must sign in and no guest access is permitted. To sign in, as shown, users may be requested to enter in their email address and password.

In other examples, scanning or interacting with communication interfaces may present an appealing and interactive splash screen (not shown) displaying a company and application's name and logo. This screen may appear for 2-3 seconds on a display of the electronic device. After the splash screen, users may be directed to the sign-up (not shown) or sign-in screen 900. If an individual wants to sign up, the individual may be directed to a sign-up screen. The following fields may be displayed and mandated to complete signup: full name; phone number (requiring OTP verification); email address (requiring OTP verification); city and country (country dropdown selection will generate a dropdown of all the cities in the country); and privacy policy, disclaimer and licensing agreement. After signing up, users may proceed to the sign-in screen. Once created, users may have unique profiles. Such profiles may track puzzles created, puzzled solved, email-addresses, and phone numbers. However, other information may be collected as well.

Once signed in, a user may be brought to a system menu 900 and may be permitted to play Irrational Squares or otherwise access the functionality of system 120/156. Depending on the physical object scanned, a system menu may vary and present a user with any number of different options, e.g., cooking recipes, nutritional information, stories, chore lists, tasks, puzzle patterns, etc.

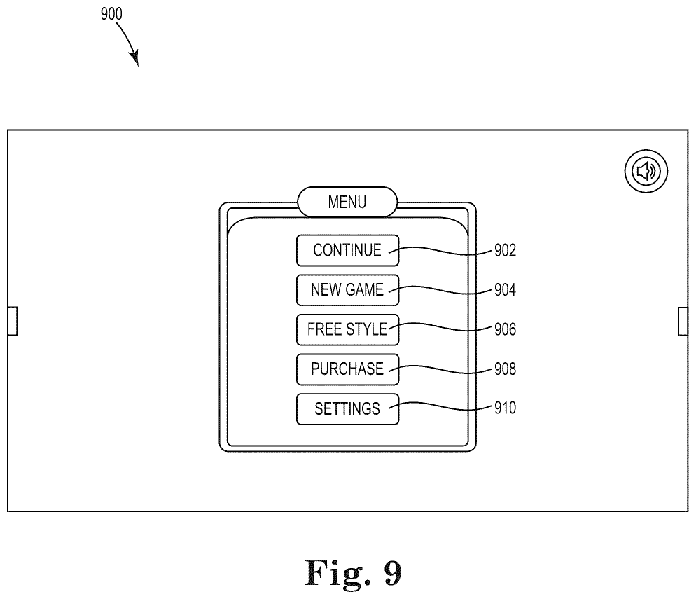

FIG. 9 illustratively shows a puzzle game menu 900 in accordance with an example of the present invention. As shown, a puzzle game menu 900 may allow a user to resume a previously started puzzle where they left off (“Continue Last Game”) 902, start a new puzzle 904 (“New Game”), create a puzzle pattern or shapes 906 (“Free Style”) with the flexibility to craft shapes according to their preferences with all the pieces, e.g., 30 pieces, displayed on the web screen, purchase or download a previously completed puzzle 908, or modify a setting of system 120/156. However, other features may be displayed as well.

For example, either by itself, or using behavioral modification system 120/156, Irrational Squares may be adapted for multi-player format-both physically and virtually. In this example, a multiplayer setting may be selected from menu 900. Once selected, system 120/156 may display unique information relating to two or more users. For example, system 120/156 may display an individual's progress or messages unique to each player.

In one example, if a user chooses “Free Style” 906, users may have the flexibility to craft shapes according to their preferences with all thirty pieces. In this mode, users may feel empowered to design any shape they desire, giving a personalized and creative touch to their experience. Once finished, users may submit their custom created shapes with details like puzzle author, puzzle name, puzzle description, and puzzle ID to an administrator for review. If approved, an administrator may seamlessly integrate the custom shape into the puzzle library, expanding the repertoire of available shapes for other users to enjoy.

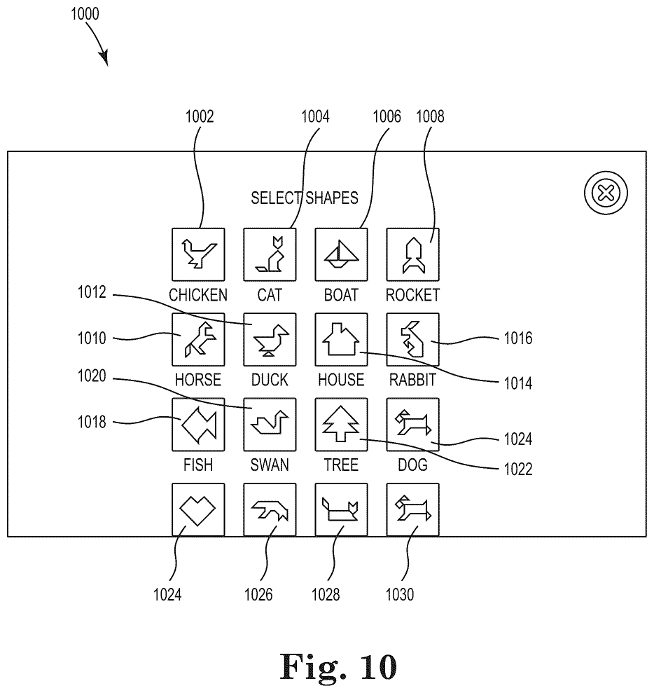

However, if a user has selected “New Game” 904, system 120/156 may present a user with any number of different puzzle patterns. For example, FIG. 10 illustratively shows administrator approved puzzle patterns in accordance with an embodiment of the present invention. As shown, patterns 1000 may include a chicken 1002, a cat 1004, a boat 1006, a rocket 1008, a horse 1010, a duck 1012, a house 1014, a rabbit 1016, a fish 1018, a swan 1020, a tree 1022, a dog 1024, a heart 1024, and shapes 1026, 1028, and 1030. However, other patterns may be selected as well.

In some examples, a puzzle pattern may be based on obtained, uploaded, or other user provided data, e.g., photos, direct inputs, etc. Alternatively, a pattern may be based on user written or spoken descriptions. It is contemplated that system 120/156 may provide an unlimited number of new puzzles and configurations, ensuring that the game remains fresh and engaging. Further, system 120/156 may also adapt the complexity of the patterns based on the user's skill level. Through system 120/156, Irrational Squares may serve as a dynamic educational tool. Specifically, it may help players develop spatial awareness, geometric understanding, and problem-solving skills.

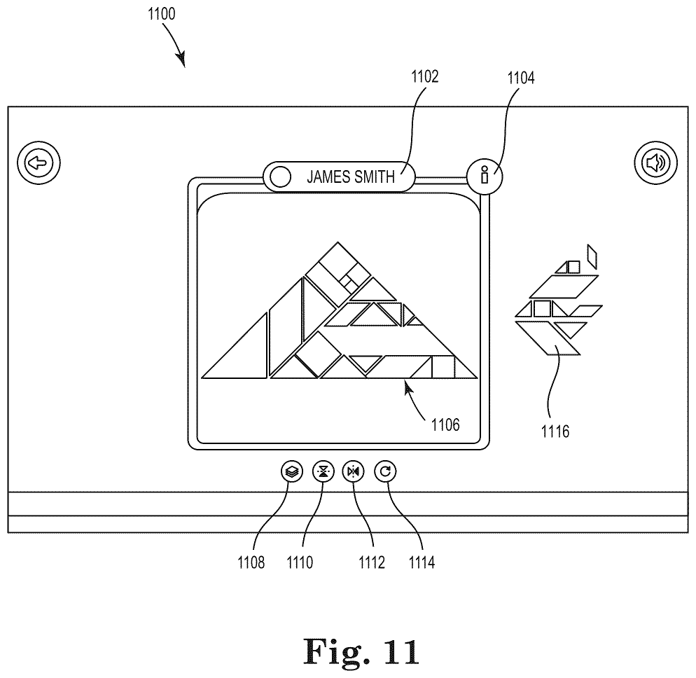

Once a particular pattern is selected, system 120/156 may present a user with a digital puzzle as shown in FIG. 11. A digital puzzle may follow the same rules as set forth above. Namely, a user may be tasked with orienting irrational puzzle pieces in different ways to complete the selected pattern. In some examples, users may be tasked with rotating or flipping individual pieces to fit a selected pattern. Rotation may be carried out in the form of a rotation wheel which may dragged to rotate the shape. Such wheel may work in 5-degree increments. Flipping may be carried out using a mirror image of the displayed shape.

As shown, system 120/156 may display all thirty, twenty, or ten puzzle pieces 1116, e.g., triangles, parallelograms, squares, long rectangles, short rectangles, etc., within a puzzle pattern 1106, author or puzzle identifying descriptions 1102, 1104, a timer with start and stop functionality 1110 and 1112, and an undo feature 1114 for removing previously placed piece(s). Additional functionality, e.g., hints, nudges, etc., may be provided as well.

To place pieces from the tray into pattern 1106, the pieces may be dropped onto the displayed pattern. To fit inside the pattern, each piece can be flipped or rotated. Once completed, the puzzle may be automatically submitted and marked as “Solved”. Additionally, in some examples, a congratulatory message, fireworks, and jolly sounds may also be provided. Until all the pieces are properly placed, such puzzle may be marked “Incomplete”.

As a user proceeds in placing the pieces, an encouraging message with an acknowledgement button may be displayed. Each game may display an encouraging message in the form of five to six messages. Encouragement messages may be displayed any time the application or system 120/156 is open. Messages may be selected from any number of messages from a message library and generated by message generation logic, e.g., “Good-job”, “Keep-going”, etc. In one example, such messages and acknowledgment buttons may be built into the codebase of the game. Further, messages may be automatically or manually displayed based on a received user input and the content and timing of a displayed message may largely depend on the physical object. For example, in Irrational Squares, such messages may be generated automatically as a user rotates or moves each individual piece. In operation, such timing may be carried out and tracked through timing logic of system 120/156. Timing logic may track the time between puzzle piece picks, total completion time of a puzzle, etc.

As a user works to complete a digital or physical puzzle, system 120/156 may generate or otherwise send “nudges” or other messages. Nudges may be created or facilitated by real-time data on a user's game play such as time between each move, time elapsed since the beginning of the game, total time played, the player's average time to completion, etc. In some examples, nudges can be designed in part to encourage players to interact more with each other to strengthen social bonds. Alternatively, nudges may also challenge players to think outside the box or approach problems in new ways.

Nudges may be powerful tools for encouraging positive behavior change. By leveraging data on an individual player's behavior and preferences, these nudges can deliver timely and relevant encouragement or reminders that resonate more effectively within each player. In other examples, nudges may be customizable. For example, users can indicate which track of nudges they prefer to receive, including any or all of Collaboration, Persistence, Creativity, Connection, etc.

Nudges may be created or otherwise supplied to system 120/156 by a trained behavioral scientist using a scoping review of literature to determine the main behavioral determinants (facilitators and barriers) of the behavior change in question. Having derived main behavioral determinants, the behavioral scientist may leverage the Theory and Techniques tool to leverage evidence-based Mechanisms of Action through which a Behavior Change Technique can be applied in the nudges. Each nudge may address a single behavioral determinant leveraging a single Behavior Change Techniques. Additionally, a behavioral scientist may train a large language model (LLM) with content-specific knowledge and deliberate prompting to produce nudges.

Nudges may also include memes and symbols re-triggering past lessons and experiences. This may serve to reinforce past lessons and experiences making the system and lessons more resilient and harder to omit or forget. Scientists may develop such algorithms or methods for developing and choosing symbols and memes for any given occasion/communication. This may be carried out through feedback/message generating logic 294.

Once a user has completed a puzzle, in some examples, a user may also be able to purchase their creations as shown in FIG. 9. For example, upon selecting the “Purchase” feature 908, a product listing may display their created works, popular works, or any other form of work. With respect to each work, a user may view details such as product name, product description, product specification, product price, and product images. If a user wants to proceed in purchasing the work, the user may have the option to add the product to their cart and place an order. The cart may be viewed before checking out and facilitate completion of the transaction. The application may also accept a wide variety of payment methods such as credit cards, debit cards, etc. In some examples, users may also have the option to view an entire list of previously ordered products and past orders.

In some examples, system 120/156 may also be able to assist with the physical completion of a puzzle pattern as well. For example, using a unique puzzle or game identifier associated with each wireless communication interface, system 120/156 may track or otherwise assist in the completion of a desired puzzle pattern. In this example, each wireless interface may allow for a seamless connection between each physical puzzle piece and its digital counterpart. This connection may allow for real-time tracking and replication of the physical puzzle layout in a digital environment to create an immersive and integrated experience. Based on a puzzle piece's orientation, positioning, or unique identifier, system 120/156 may provide immediate feedback in the way of suggestions, encouragement, or other helpful feedback.

The digital representation or mirror image of each puzzle piece may be displayed on a display of an electronic device, e.g., display 142 of electronic device 106. As a user modifies a piece's position or orientation, system 120/156 may reflect the change simultaneously on the display of an electronic device. This may allow for a dynamic and engaging experience.

Additionally, the wireless interface may also trigger specific educational content or challenges as well based on a type of selected piece and its orientation and positioning. For example, when a user creates a particular shape, system 120/156 may provide historical information about that shape, offer additional challenges, or unlock new content. Further, after identifying each piece, system 120/156 may track a user's progress over time. This data can be used to personalize the difficulty level of challenges, suggest new patterns based on skill level, or provide tailored encouragement messages, all from system 120/156, enhancing a user's learning curve and experience.

Further, wireless interfaces may facilitate the sharing of user-created patterns. In this example, users can create their own designs, which, once recognized by system 120/156, can be saved and shared with the broader community through system 120/156. In other examples, the wireless interfaces may make the setup process for the digital twin or mirror image straightforward. Users may simply arrange the physical pieces and system 120/156 automatically replicates the layout.

Additionally, system 120/156 may also allow players to interact with the puzzle across various devices and platforms. For example, players may use a mobile app, e.g., behavioral modification system 120, for on-the-go access or a web interface for a more comprehensive experience on larger screens. In one example, system 120/156 provides a more immersive and interactive experience by bridging the physical pieces with their virtual counterparts. Players can arrange the physical pieces and see their efforts mirrored in the digital world in real-time. This integration can also include augmented reality features, enhancing the tactile experience with virtual enhancements.

System 120/156 may also connect players from around the world allowing them to share their designs, compete in challenges, and collaborate on puzzles. Moreover, system 120/156 may incorporate educational content, tutorials, and challenges that adapt to a player's skill level. Moreover, an application, e.g., application 120, may track progress over time, providing insights and feedback that encourages continued learning and improvement.

System 120/156 may also offer personalized experiences based on user behavior and preferences. Features like customized puzzles, difficulty levels, and personalized encouragement messages can keep players engaged and motivated. Additionally, valuable user data can be collected, such as common puzzle choices, difficulty levels, and time spent on certain tasks. This data can be used to improve the game, tailoring it to user preferences and introducing new features that align with player desires and behaviors.

In one example, a created digital twin or mirror image can be created and manipulated using finger movements and gestures. This may allow players to see a virtual representation of their physical creations. This feature may enhance interactivity, as changes made to the physical puzzle are instantly reflected in the digital environment via the wireless communication interfaces, e.g., interface 164, making the gameplay experience more dynamic and engaging. Players may also share their digital puzzle configurations with others remotely, allowing for collaborative problem-solving and creativity. This feature may prove particularly beneficial in educational or family settings when family members are in different locations.

System 120/156 may also allow for personalized feedback based on a player's actions with the physical puzzle. It can offer suggestions, show alternative solutions, or provide hints tailored to the player's skill level and progress.

Through system 120/156, user-provided content may also be directly or indirectly provided and used to evolve the system. This may bring a wealth of diverse perspectives and ideas. Players from different backgrounds and with varying skill levels can offer unique insights and creative solutions that might not be immediately apparent to other players. This may further encourage creativity and personal expression. The shared content may become a source of inspiration and variety, keeping the game fresh and dynamic, and contribute to building a strong community. Through system 120/156, players can connect over shared interests, compete in user-created challenges, or collaborate on complex designs. Players may be exposed to a multitude of configurations, each offering unique problem-solving experiences. This variety may cater to a wide range of skill levels and interests.

User provided content may include, but is not limited to, drawings (e.g., illustration, diagram, picture, rendering, design, draft, pattern, shape, solution, etc.), recipes (e.g., formula, algorithm, instructions, guide, blueprint, method, plan, guidelines, directions, procedures, steps, etc.), poems (e.g., verse, ode, lyric, rhyme, epic, etc.), etc.

In one example, “Irrational Squares” and the services of system 120/156 may be implemented through a web-based application. For example, such application may be created through HTML, CSS, LARAVEL, or a 3.JS/HTML 5 technology stack. The application may be accessed by general users, serve as an ecommerce site, and allow for special administrative privileges.

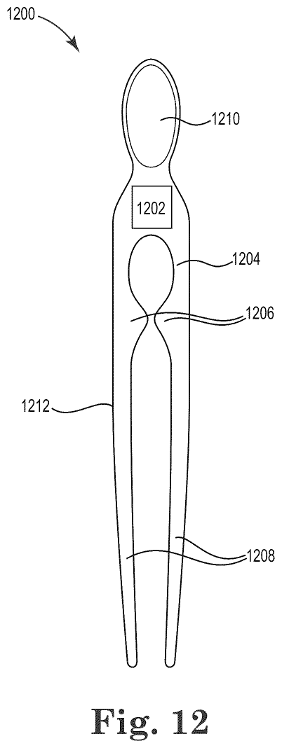

FIG. 12 illustratively shows a utensil in accordance with an example of the present invention. As noted above, a utensil 1200 may be used in the preparation, consumption, or transportation of food or other material. As shown, utensil 1200 illustratively includes a uniform body 1212 attached to a wireless communication interface 1202. Body 1212 may be formed from, for example, wool, plastic, or metal and include an indented or curved top end 1210, an opening 1204 mirroring a shape of top end 1210, movement restrictors 1206, and two elongated members 1208. In operation, either members 1208 or top end 1210 may be used in the preparation, consumption, or transportation of food or other material.

For example, a user may compress or otherwise squeeze together members 1208 to grab and transport material. Such compression may be facilitated through the incorporation of flexible opening 1204 with restrictors 1206 preventing an overextension of members 1208. Specifically, as members 1208 compress, restrictors 1206 may simultaneously compress and come together before, or about the same time as, member ends 1208 to prevent a breaking of members 1208. Alternatively, a user may simply use indented or curved top end 1210 to pick up food or other material.

Utensil 1200 dimensions may vary depending on a desired operation and utensil size. However, in exemplary examples, utensil 1200 may be about 11 inches long and 1.5 inches wide. A total length of utensil 1200 may vary between 3-15 inches with shorter lengths, e.g., between 3-8 inches, being used for the transportation of smaller materials or snacks, medium lengths, e.g., between 8-13 inches, being used for the transportation of larger material or food, and longer lengths, e.g., between 13-18 inches, being used for the cooking, preparation, or serving of food or material.

As noted above, wireless communication interface 1202, e.g., NFC tag, may couple to body 1212 and allow for user interaction with a behavior modification system, e.g., system 120/156. In some examples, interface 1202 may include a unique identifier such that, when read using an electronic device, e.g., device 106, system 120/156 may identify the unique identifier and display personalized information which may include healthy recipes, uploaded and shared recipes of previous meals, and healthy habit positive nudge messages encouraging users to maintain or pursue healthy eating habits and recommended healthy recipes.

However, in some examples, advertisements, words, or other marks may be attached or engraved near interface 1202. Such markings may allow for company identification, system instructions, or any other information.

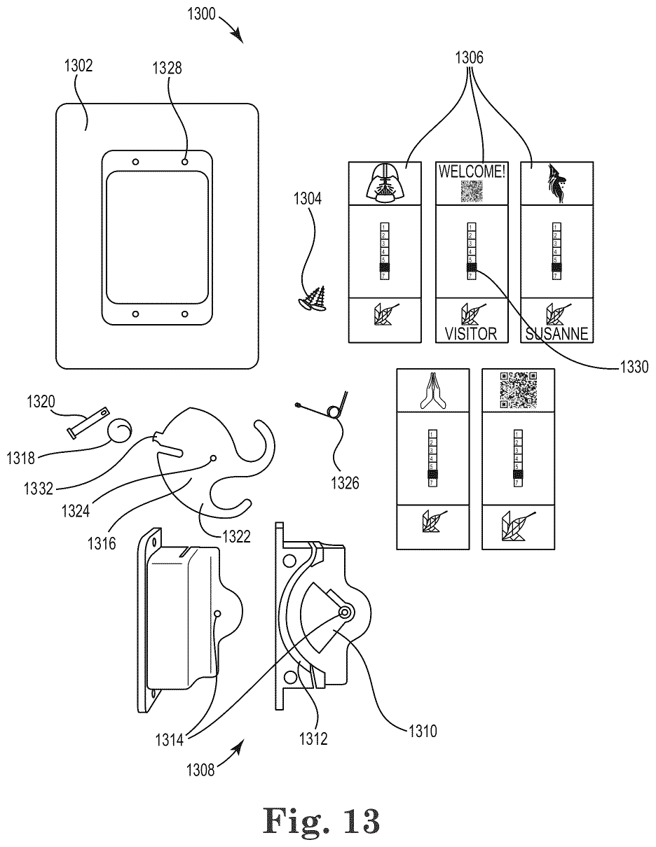

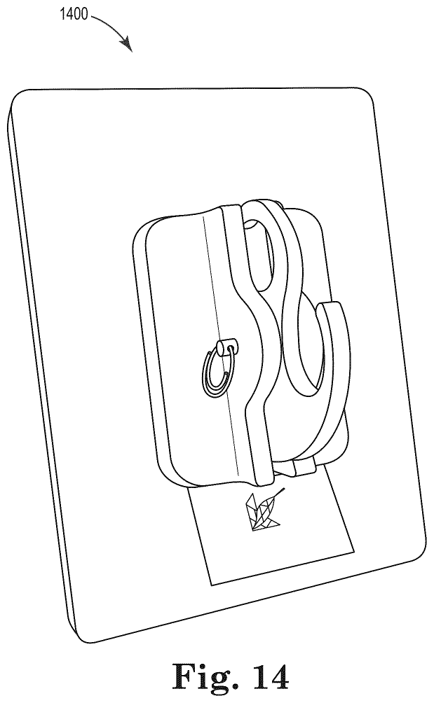

FIGS. 13-14 illustratively show a hanger assembly in accordance with examples of the present invention. As shown, a hanger assembly 1300 may include a cotter pin 1320 and ring 1318 for fastening together the components of assembly 1300, one or more nudge cards 1306 for displaying information and a communication interface, a rotatable hook body 1316 for hanging or otherwise storing a clothing article and displaying information on card 1306, a biasing member 1326, e.g., spring, for biasing hook body 1316 to a first, upright position, a panel 1302 for securely fastening components of assembly 1300 to a mounting surface, e.g., wall, a two-part housing 1308 for enclosing all, or part of, biasing member 1326, hook body 1316, pin 1320, and card 1306, and one or more fastening members 1304 for securing panel 1302 to a mounting surface and housing 1308 to panel 1302.

Rotatable hook body 1316 may include one or more hooks 1322 for receiving and storing a clothing article, e.g., shirt, coat, hat, etc., apertures 1324 for receiving cotter pin 1320, and protrusion(s) 1332 for coupling hook body 1316 to card 1306. Rotatable hook body 1316 may be securely fastened in-between two-part housing 1308 as cotter pin 1320 passes through apertures of housing and hook body 1314 and 1324, respectively.

In operation, once a clothing article is positioned on one or more hooks 1322, a weight of the clothing article may cause hook body 1316 to rotate downwards to a second storage position. In turn, protrusion 1332 may be elevated or otherwise raised to place card 1306 at a second display position as protrusion passes through one or more apertures 1330 of card 1306. At a first or second display position, card(s) 1306 may display one or more communication interfaces, e.g., NFC tag, Bluetooth low energy (BLE), etc., or messages, e.g., “welcome home”, “thank you for hanging your coat”, “you are just in time”, etc. In one example, communication interfaces on cards 1306 may allow system 120/156 to display a calendar, chore list, progress report, nudge messages promoting good healthy habits, etc. on a display of an electronic device, e.g., display 142. Once a clothing article is removed from hook(s) 1322, hook body 1316 may rotate or return to a first operating position as biasing member 1326 acts on hook body 1316 within housing 1308.

Two-part housing 1308 illustratively includes a slot 1312 for receiving and housing card(s) 1306, apertures 1314 for receiving cotter pin 1320, and biasing member area 1310 for positioning member 1326. Housing 1308 may connect to panel 1302 using any number of fastening member(s) 1304 and apertures on housing (not shown) and panel 1328. In turn, panel 1302 may connect to a mounting surface using apertures 1302 and fastening members 1304 as illustratively shown in FIG. 14.

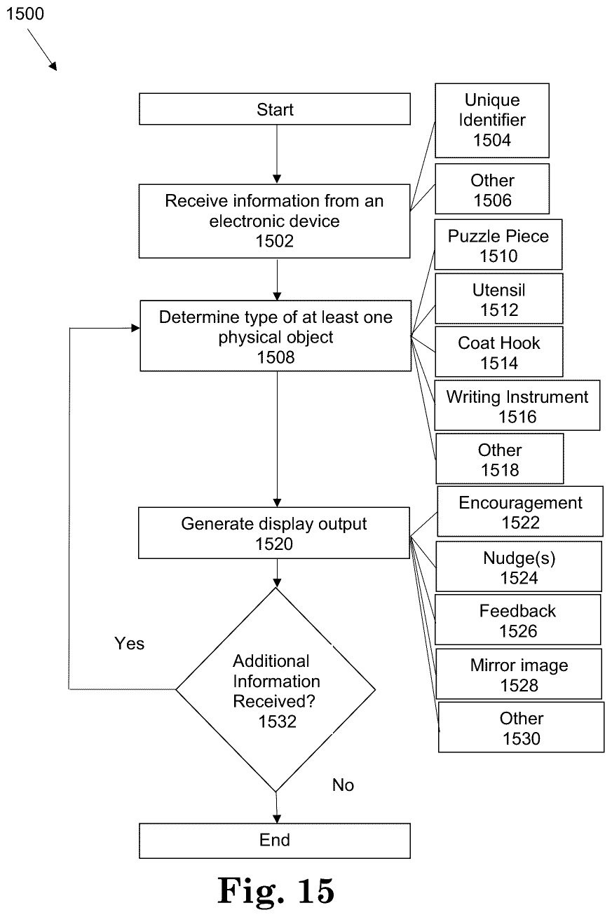

FIG. 15 illustratively shows a method of displaying encouragement, nudge messages, feedback, or other information in accordance with an example of the present invention. As shown, method 1500 begins at block 1502 where a behavior modification system, e.g., system 120/156, receives information associated with a type of physical object 1504 from an electronic device communicatively coupled to a physical object. While information may indicate a type of object, other information 1506 may be communicated as well, e.g., user profile information, operational information, etc.

Once received, method 1500 proceeds to block 1508 where a type of the physical object is determined based on the information. A physical object may be identified as, for example, a puzzle piece 1510 (or a plurality of puzzle pieces) utensil 1512, a coat hook 1514, a writing instrument 1516, or other physical object 1518. After which, method 1500 proceeds to block 1520 where a display output is generated based on the type of physical object. A display output may include encouragement 1522, nudge message(s) 1524, feedback 1526, mirror images of the physical object 1528, or other messages 1520. Once displayed, method 1500 proceeds to block 1532 where a behavior modification system determines whether additional information is being received from an electronic device communicatively coupled to a physical object. If yes, method 1500 reverts to block 1508 where a type of object is determined. If not, method 1500 ends.

The above description is directed to the disclosed methods and is not intended to limit them. Those of skill in the art will readily appreciate that the teachings found herein may be applied to yet other embodiments within the scope of the attached claims. The complete disclosures of all cited patents, patent documents, and publications are incorporated herein by reference as if individually incorporated. However, in case of any inconsistencies the present disclosure, including any definitions herein, will prevail.

1. An interactive system for developing positive behavioral traits comprising:
at least one physical object coupled to a wireless communication interface configured to communicatively couple to an electronic device; and
a behavioral modification system configured to communicatively couple to the electronic device, to receive information associated with the wireless communication interface from the electronic device, and, based on the received information, to generate and display at least one of encouragement messages, nudge messages infused with behavioral change techniques, feedback, or user provided content on a display of the electronic device.

2. The interactive system of claim 1, wherein the wireless communication interface comprises an NFC tag, RFID tag, or BLE.

3. The interactive system of claim 2, wherein the electronic device comprises a tablet, mobile device, desktop computer, or laptop computer.

4. The interactive system of claim 3, wherein the at least one physical object comprises at least one puzzle piece sized and dimensioned in accordance with an irrational number, and wherein the puzzle piece is configured to be placed within a pattern.

5. The interactive system of claim 4, wherein the irrational number comprises a square root of two or a golden ratio.

6. The interactive system of claim 3, wherein the at least one physical object comprises a cooking utensil comprising a body with an indented top end, flexible opening, movement restrictors, and elongated members for preparing, consuming, or transporting food or other material.

7. The interactive system of claim 3, wherein the at least one physical object comprises a writing instrument.

8. The interactive system of claim 3, wherein the at least one physical object comprises a nudge card configured to couple to a hook assembly with a rotatable hook body operable in a first upright position and a second storage position, wherein in the first position, the nudge card is positioned in a first display position and, in the second position, a second display position.

9. The interactive system of claim 6, wherein the user provided content comprises at least one of cooking information, recipes, or calorie management information.

10. The interactive system of claim 7, wherein the user provided content comprises at least one of stories, misinformation and information, fables, or writing samples.

11. The interactive system of claim 8, wherein the nudge message indicates a presence of a user based on a placement of a clothing article on the rotatable hook body.

12. The interactive system of claim 5, wherein the feedback comprises a mirror image of the at least one puzzle piece and pattern.

13. An interactive system for developing positive behavioral traits, comprising:
a puzzle comprising a plurality of puzzle pieces sized and dimensioned based on at least one irrational ratio configured to be positioned with a puzzle pattern, wherein at least one puzzle piece comprises a wireless communication interface; and
a behavioral modification system communicatively coupled to the least one puzzle piece configured to receive a unique identifier associated with the puzzle and, based on the identifier, to generate and display at least one of encouragement messages, nudge messages infused with behavioral change techniques, feedback, or a mirror image of the puzzle on a display of an electronic device.

14. The interactive system of claim 13, wherein the plurality of puzzle pieces comprises squares, triangles, and parallelograms sized and dimensioned based on the irrational ratio.

15. The interactive system of claim 14, wherein the at least one irrational ratio comprises a plurality of irrational ratios, the plurality of irrational ratios comprising a square root of two and a golden ratio, and wherein a first puzzle piece is sized and dimensioned based on the square root of two and a second puzzle piece is sized and dimensioned based on the golden ratio.

16. A method of eliciting positive behavioral change, comprising:
receiving information from an electronic device communicatively coupled to at least one physical object, the information including at least a unique identifier associated with the type of at least one physical object;
determining the type of at least one physical object based on the received unique identifier; and
generating a display output for a display device of the electronic device based on the type of at least one physical object, wherein the display output comprises at least one of encouragement messages, nudge messages infused with behavioral change techniques, feedback, or a mirror image.

17. The method of claim 16, wherein the at least one physical object comprises at least one of a puzzle piece, a hook, a cooking utensil, and a writing instrument coupled to a wireless communication interface comprising the unique physical object identifier.

18. The method of claim 17, wherein the at least one physical object comprises the puzzle piece sized and dimensioned based on an irrational ratio, wherein the irrational ratio comprises a square root of two or a golden ratio.

19. The interactive system of claim 1, wherein the at least one physical object comprises at least one puzzle piece sized and dimensioned based on at least one irrational ratio comprising a square root of two or a golden ratio, the wireless communication interface comprises an NFC tag, and the behavior modification system comprises:
puzzle pattern generation logic;
digital twin generation logic;
a digital gameplay module; and
a free style gameplay module,
wherein said logic and modules are stored in a non-transitory data storage accessible over a network through the electronic device.

20. The interactive system of claim 1, wherein the at least one physical object comprises a utensil comprising a body comprising an indented top end, flexible opening, movement restrictors, and elongated members, and wherein the wireless communication interface comprises an NFC tag, the behavior modification system comprises recipe generation logic, calorie management logic, utensil tracking logic, and a multi-player module, wherein said logic and modules are stored in a non-transitory data storage accessible over a network through the electronic device.
